(19) United States
(12) Patent Application Publication
(10) Pub. No.: US 2016/0330554 A1
(43) Pub. Date: Nov. 10, 2016

(54) LOCATION-BASED SELECTION OF PROCESSING SETTINGS

(71) Applicant: Martin Evert Gustaf Hillbratt, Molnlycke (SE)

(72) Inventor: Martin Evert Gustaf Hillbratt, Molnlycke (SE)

(21) Appl. No.: 15/009,011

(22) Filed: Jan. 28, 2016

Related U.S. Application Data
(60) Provisional application No. 62/158,617, filed on May 8, 2015.

(51) Int. Cl. H04R 25/00; A61N 1/36

(52) U.S. Cl. CPC H04R 25/558; A61N 1/36032; H04R 25/305; H04R 2225/41

(57) ABSTRACT

Embodiments presented herein are generally directed to techniques for the selection of processing settings based on a location of a medical device. The techniques presented herein define an aggregate mapped area for a medical device and one or more processing settings are selected for use by the medical device when the medical device is correlated with the aggregate mapped area.

### CROSS-REFERENCE TO RELATED APPLICATIONS

This application claims priority to U.S. Provisional Application No. 62/158,617 entitled “Location-Based Selection of Processing Settings,” filed May 8, 2015, the content of which is hereby incorporated by reference herein.

### BACKGROUND

1. Field of the Invention

The present invention relates generally to selection of medical device settings.

2. Related Art

Hearing loss, which may be due to many different causes, is generally of two types, conductive and/or sensorineural. Conductive hearing loss occurs when the normal mechanical pathways of the outer and/or middle ear are impeded, for example, by damage to the ossicular chain or ear canal. Sensorineural hearing loss occurs when there is damage to the inner ear, or to the nerve pathways from the inner ear to the brain.

Individuals who suffer from conductive hearing loss typically have some form of residual hearing because the hair cells in the cochlea are undamaged. As such, individuals suffering from conductive hearing loss typically receive an auditory prosthesis that generates motion of the cochlea fluid. Such auditory prostheses include, for example, acoustic hearing aids, bone conduction devices, and direct acoustic stimulators.

In many people who are profoundly deaf, however, the reason for their deafness is sensorineural hearing loss. Those suffering from some forms of sensorineural hearing loss are unable to derive suitable benefit from auditory prostheses that generate mechanical motion of the cochlea fluid. Such individuals can benefit from implantable auditory prostheses that stimulate nerve cells of the recipient's auditory system in other ways (e.g., electrical, optical and the like). Cochlear implants are often proposed when the sensorineural hearing loss is due to the absence or destruction of the cochlea hair cells, which transduce acoustic signals into nerve impulses. An auditory brainstem stimulator is another type of electrically-stimulating auditory prosthesis that might also be proposed when a recipient experiences sensorineural hearing loss due to damage to the auditory nerve.

### SUMMARY

In one aspect a method is provided. The method comprises: defining an aggregate mapped area for a medical device, wherein the aggregate mapped area is a digital representation of a spatial region, and wherein the spatial region is defined through the analysis of digital map data and is associated with a selected location point; selecting one or more processing settings for use by the medical device when situated in the aggregate mapped area; determining that the medical device is at least one of positioned within, substantially positioned within, or is anticipated to be positioned within the aggregate mapped area; and activating the one or more processing settings in response to the determining that the medical device is at least one of positioned within, substantially positioned within, or is anticipated to be positioned within the aggregate mapped area.

In another aspect a medical device system is provided. The medical device system comprises: a medical device; and a computing device comprising a memory and one or more processors configured to: determine a location for the medical system; determine, based on the location of the medical system and map data, that the medical system is at correlated with an aggregate mapped area; and activate one or more settings for the medical system in response to determining that the medical system is correlated with the aggregate mapped area.

In another aspect a system is provided. The system comprises: a memory; and one or more processors configured to: determine a selected map location for a medical device, define an aggregate mapped area for the medical device, wherein the aggregate mapped area is a digital representation of a spatial region, and wherein the spatial region is defined through the analysis of digital map data and is associated with the selected map location, and select one or more processing settings for use by the medical device when situated in the aggregate mapped area.

### BRIEF DESCRIPTION OF THE DRAWINGS

Embodiments of the present invention are described herein in conjunction with the accompanying drawings, in which:

FIG. 1 is a schematic diagram illustrating a cochlear implant system in accordance with embodiments presented herein;

FIG. 2 is a flowchart illustrating a method for location-based selection of sound processing settings in accordance with embodiments presented herein;

FIG. 3 is a simplified view of a digital map that may be used in embodiments presented herein;

FIG. 4 is a flowchart illustrating a method for defining an aggregate mapped hearing area in accordance with embodiments presented herein;

FIGS. 5A-5D are schematic diagrams illustrating steps for defining an aggregate mapped hearing area in accordance with embodiments presented herein;

FIG. 6 is a block diagram of a mobile computing device configured to perform all or part of the techniques for location-based selection of sound processing settings in accordance with embodiments presented herein; and

FIG. 7 is a block diagram of a fitting system configured to perform all or part of the techniques for location-based selection of sound processing settings in accordance with embodiments presented herein.

### DETAILED DESCRIPTION

Embodiments presented herein are generally directed to techniques for the selection of processing settings based on a location of a medical device. The techniques presented herein define an aggregate mapped area for a medical device and one or more processing settings are selected for use by the medical device when correlated with the aggregate mapped area.

Merely for ease of description, the techniques presented herein for location-based selection of processing settings are primarily described herein with reference to an illustrative medical device, namely a cochlear implant. However, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used with other hearing prostheses, including acoustic hearing aids, bone conduction devices, middle ear auditory prostheses, direct acoustic stimulators, other electrically simulating auditory prostheses (e.g., auditory brain stimulators), etc. The techniques presented herein may also be used with visual prostheses (i.e., Bionic eyes), sensors, pacemakers, drug delivery systems, defibrillators, functional electrical stimulation devices, catheters, etc. In further embodiments, the techniques presented herein may be used with air purifiers or air sensors (e.g., automatically adjust depending on environment), hospital beds, identification (ID) badges/bands, or other hospital equipment or instruments.

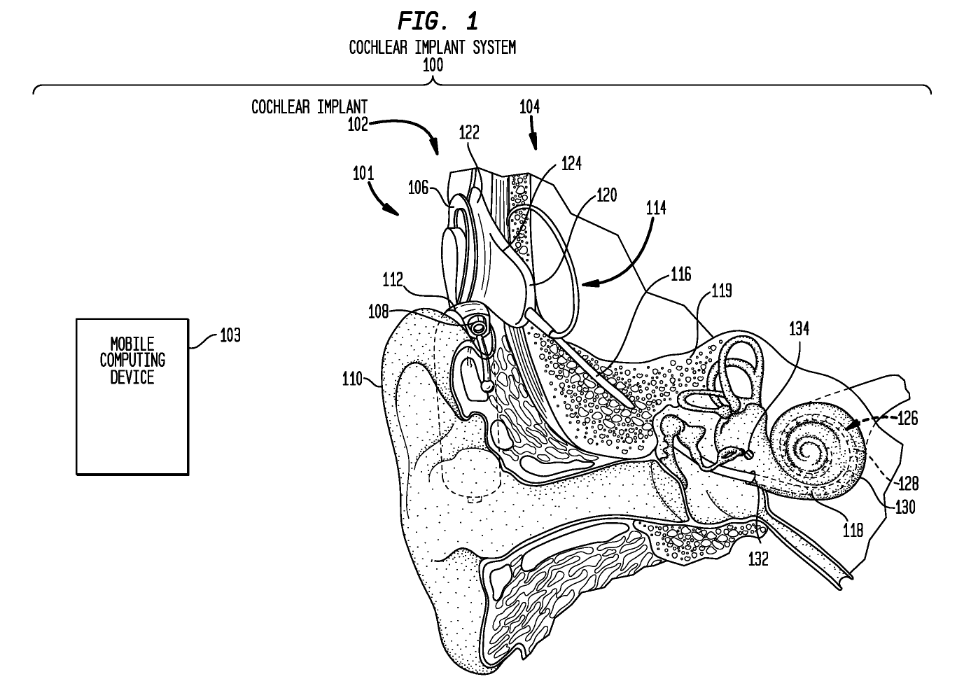

FIG. 1 is perspective view of an exemplary medical device system 100, namely a cochlear implant system, in which the techniques for location-based selection of processing settings presented herein are implemented. The cochlear implant system 100 comprises a cochlear implant 102 and a mobile computing device 103.

The cochlear implant 102 includes an external component 101 and an internal or implantable component 104. The external component 101 is directly or indirectly attached to the body of the recipient and typically comprises an external coil 106 and, generally, a magnet (not shown in FIG. 1) fixed relative to the external coil 106, one or more sound input elements 108 (e.g., microphones, telecoils, etc.) for detecting sound and a sound processing unit 112. The sound processing unit 112 may include, for example, a power source (not shown in FIG. 1) and a sound processor (also not shown in FIG. 1). The sound processor is configured to process electrical signals generated by a sound input element 108 that is positioned, in the depicted embodiment, by auricle 110 of the recipient. The sound processor provides the processed signals to external coil 106 via a cable (not shown in FIG. 1).

FIG. 1 illustrates an example in which cochlear implant 102 includes an external component 101 with an external sound processor. It is to be appreciated that the use of an external component is merely illustrative and that the techniques presented herein may be used in arrangements having an implanted sound processor (e.g., totally implantable cochlear implants). It is also to be appreciated that the individual components referenced herein, e.g., sound input element 108 and the sound processor in sound processing unit 112, may be distributed across more than one tissue-stimulating prosthesis, e.g., two cochlear implants 102, and indeed across more than one type of device, e.g., cochlear implant 102 and a consumer electronic device or a remote control of the cochlear implant 102.

The implantable component 104 comprises an implant body 114, a lead region 116, and an elongate intra-cochlear stimulating assembly 118. The implant body 114 comprises a stimulator unit 120, an internal coil 122, and an internal receiver/transceiver unit 124, sometimes referred to herein as transceiver unit 124. The transceiver unit 124 is connected to the internal coil 122 and, generally, a magnet (not shown) fixed relative to the internal coil 122.

The magnets in the external component 101 and implantable component 104 facilitate the operational alignment of the external coil 106 with the internal coil 122. The operational alignment of the coils enables the internal coil 122 to transmit/receive power and data to/from the external coil 106. More specifically, in certain examples, external coil 106 transmits electrical signals (e.g., power and stimulation data) to internal coil 122 via a radio frequency (RF) link. Internal coil 122 is typically a wire antenna coil that is electrical insulated by a flexible molding (e.g., silicone molding). In use, transceiver unit 124 may be positioned in a recess of the temporal bone of the recipient. Various other types of energy transfer, such as infrared (IR), electromagnetic, capacitive and inductive transfer, may be used to transfer the power and/or data from an external device to cochlear implant and FIG. 1 illustrates only one example arrangement.

Elongate stimulating assembly 118 is configured to be at least partially implanted in cochlea 130 and includes a plurality of intra-cochlear stimulating contacts 128. The stimulating contacts 128 collectively form a contact array 126 and may comprise electrical contacts and/or optical contacts. Stimulating assembly 118 extends through an opening in the cochlea 130 (e.g., cochleostomy 132, the round window 134, etc.) and has a proximal end connected to stimulator unit 120 via lead region 116 that extends through mastoid bone 119. Lead region 116 couples the stimulating assembly 118 to implant body 114 and, more particularly, stimulator unit 120.

The mobile computing device 103 is a portable electronic component capable of storing and processing electronic data and configured to communicate with the cochlear implant 102. Mobile computing device 103 may comprise, for example, a mobile or satellite “smart” phone, collectively and generally referred to herein simply as “mobile phones,” a tablet computer, a personal digital assistant (PDA), a remote control device, or another portable personal device enabled with processing and communication capabilities.

As noted, cochlear implant 102 includes one or more sound input elements 108 that receive electrical signals and/or convert audio signals into electrical input signals. The sound processor processes the electrical input signals and generates stimulation data for use in delivering stimulation to the recipient in accordance with various operating parameters dictated by one of a number of selectable settings or modes of operation. The various selectable settings or modes of operation may be in the form of executable programs or sets of parameters for use in a program. The settings may accommodate any of a number of specific configurations that influence the operation of the cochlear implant. For example, the settings may include different digital signal and sound processing algorithms, processes and/or operational parameters for different algorithms, other types of executable programs (such as system configuration, user interface, etc.), or operational parameters for such programs. In certain examples, the selectable settings would be stored in a memory of the cochlear implant 102 and relate to different optimal settings for different listening situations or environments encountered by the recipient (i.e., noisy or quite environments, windy environments, etc.).

Additionally, since the dynamic range for electrical stimulation is relatively narrow and varies across recipients and stimulating contacts, programs used in a sound processor are typically individually tailored to optimize the perceptions presented to a particular recipient (i.e., tailor the characteristics of electrical stimulation for each recipient). For example, many speech processing strategies rely on a customized set of stimulation settings which provide, for a particular recipient, the threshold levels (T-levels) and comfortable levels (C-levels) of stimulation for each frequency band. Once these stimulation settings are established, the sound processor may then optimally process and convert the received acoustic signals into stimulation data for use by the stimulator unit 120 in delivering stimulation signals to the recipient.

As such, it is clear that a typical cochlear implant has many parameters which determine the sound processing operations of the device. The individualized programs, commands, data, settings, parameters, instructions, modes, and/or other information that define the specific characteristics used by cochlear implant 102 to process electrical input signals and generate stimulation data therefrom are generally and collectively referred to as “sound processing settings.” As described further below, presented herein are techniques for activation and use of one or more sound processing or other settings based on a location of the cochlear implant system 100.

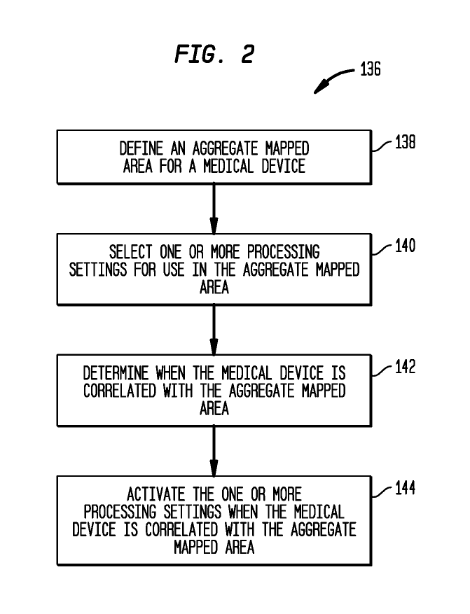

More specifically, FIG. 2 is a high-level flowchart of a method 136 for the location-based selection of sound processing settings for cochlear implant 102 in accordance with embodiments presented herein. As shown, method 136 includes a step 138 in which an “aggregate mapped hearing area” for cochlear implant 102 is defined. As described further below, an aggregate mapped hearing area is a representation of a bounded spatial/geographic region defined through the analysis of digital/electronic map data in which the cochlear implant is configured to activate and use one or more selected sound processing settings. Digital or electronic map data refers to map data converted in a computer readable/processable form. Digital map data is compiled and formatted into a virtual image (i.e., a “digital map”) for display on a computing device. The function of the electronic map data is to enable a computing device to display an accurate representation of a particular spatial area, detailing, for example, roads/streets, buildings, points of interest, etc. Digital maps (and thus the underlying digital map data) may take a number of different forms, such as satellite/aerial maps, so-called “birds-eye” maps (i.e., angled aerial maps), street/road maps, “hybrid” maps (i.e., combination of aerial and street maps), street-view maps, three-dimensional (3D) maps, etc. It is to be appreciated that an aggregate mapped hearing area in accordance with embodiments presented herein includes areas mapped using any of the above or types of digital maps and underlying digital map data.

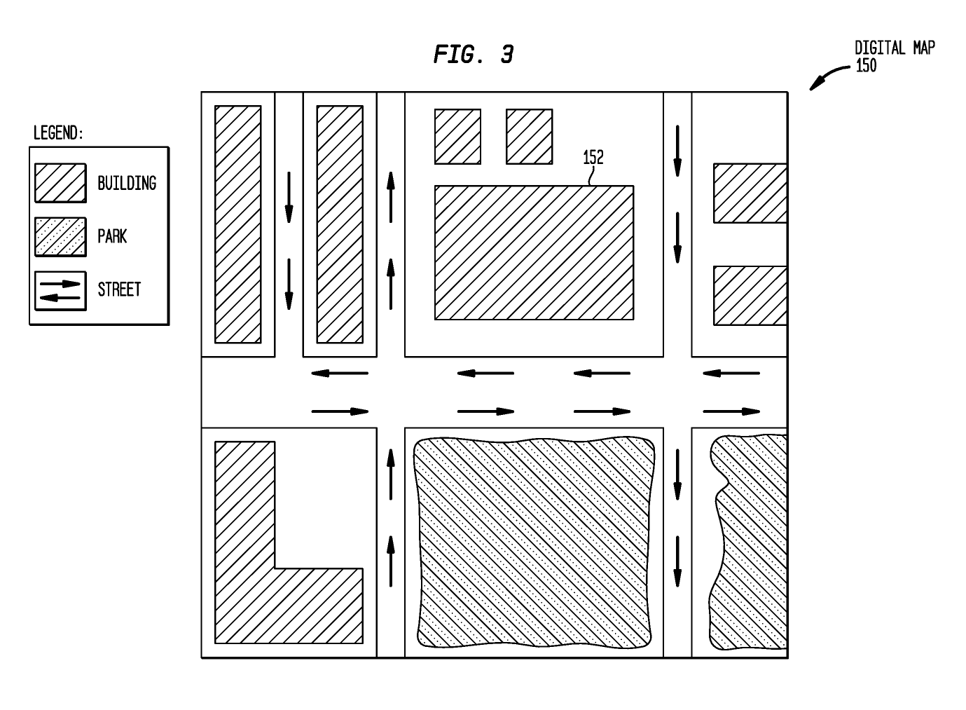

Techniques for the generation and display of digital maps are known and are not described further herein. However, FIG. 3 illustrates an example digital map 150 that may be displayed, for example, at a display screen of mobile computing device 103 using digital map data. The digital map 150 is a simplified street map that illustrates a grid of streets/roads, buildings, and parks. Merely for ease illustration, further details of the location-based selection of sound processing settings will be described herein with reference to the digital map 150 and, more particularly, with reference to a building 152 shown as part of digital map 150.

Returning to FIG. 2, method 136 further includes a step 140 in which one or more sound processing settings are selected for use in the aggregate mapped hearing area. Method 136 also includes a step 142 in which the cochlear implant system 100 determines when the cochlear implant system 100 is “correlated with” the aggregate mapped hearing area. Additionally, at step 144, the one or more sound processing settings are activated for use when the cochlear implant system 100 is positioned in the aggregate mapped hearing area.

As described further below, a cochlear implant system 100 or other medical device is “correlated with” an aggregate mapped area when the cochlear implant system or medical device is at least one of situated/positioned in/within, substantially positioned in, or anticipated to be positioned in the aggregate mapped area. In one specific example, the cochlear implant system 100 or other medical device is correlated with an aggregate mapped area when the cochlear implant system or medical device is estimated to be positioned in a specified physical proximity of the aggregate mapped area.

As described further below an aggregate mapped hearing area is a digital representation of a spatial region defined through the analysis of digital map data. It is to be appreciated that the positioning of cochlear implant system 100 “within” an aggregate mapped hearing area refers to the positioning of the cochlear implant system 100 within the spatial region corresponding to (i.e., represented/defined by) the aggregate mapped hearing area.

The order of steps 138, 140, 142, and 144 shown in FIG. 2 are illustrative and it possible for the order of the steps to change in different embodiments. Further details of each of the steps 138, 140, 142, and 144 of FIG. 2 are provided below.

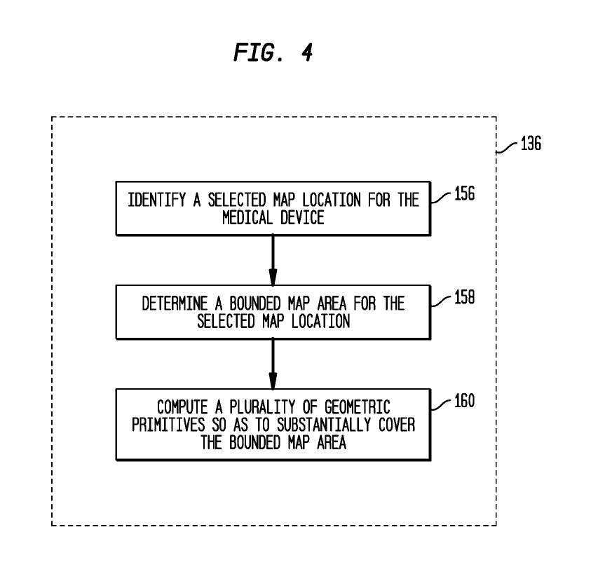

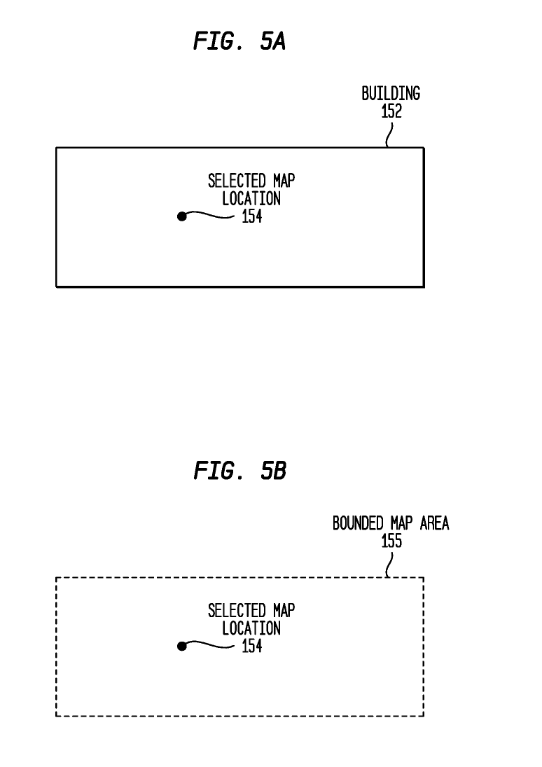

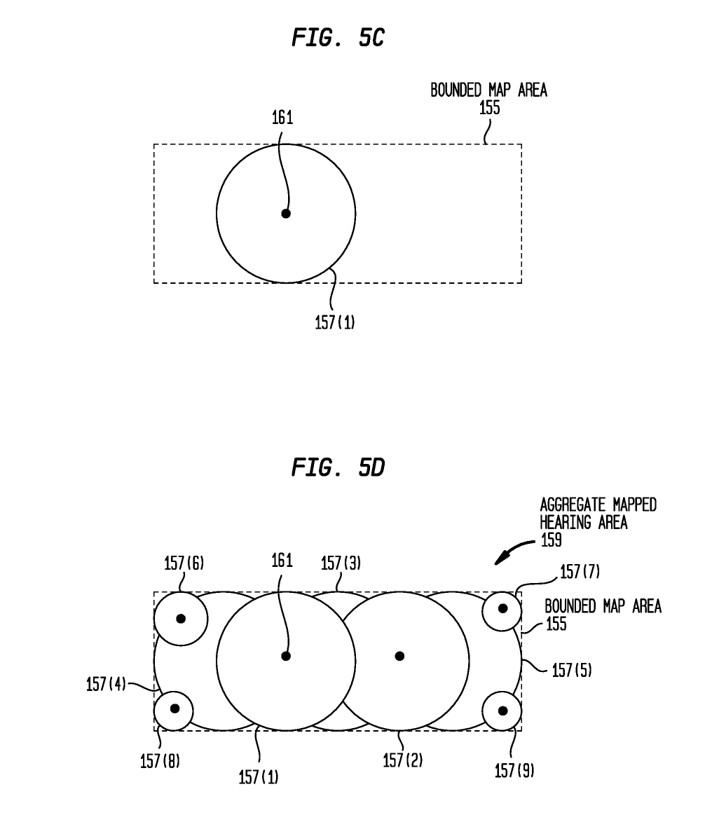

FIG. 4 is flowchart illustrating an example method that may be performed at step 138 (FIG. 2) to define an aggregate mapped hearing area for cochlear implant 102 in accordance with embodiments presented herein. FIG. 5A is a schematic diagram illustrating building 152 extracted from digital map 150 of FIG. 3.

The method of FIG. 4 begins at 156 where a selected map location for cochlear implant system 100 is identified. As used herein, a selected map location is a spatial point at which one or more sound processing settings are to be activated by the cochlear implant 102. FIG. 5A illustrates a selected map location 154 situated within building 152.

Several techniques may be used to identify a selected map location. In one embodiment, the cochlear implant system 100, more particularly, mobile computing device 103, determines a current location for the cochlear implant 102 through the use of a positioning system. The current location of the mobile computing device 103 is identified as the selected map location. The determination of the current location of the mobile computing device 103 may be triggered in response to, for example, a voice command, a user input at the mobile computing device 103 or the cochlear implant 102, etc.

For example, in one embodiment mobile computing device 103 makes use of a satellite navigation/positioning system to determine the current location of the computing device. Since the mobile computing device 103 is generally within the immediate proximity of the recipient (e.g., carried by the recipient, within a bag carried by recipient, etc.), the current location of the computing device also represents the current location of the cochlear implant 102.

Satellite positioning systems are known in the art and are not described in detail herein. However, it is to be appreciated that embodiments presented herein may make use of any of a number of different satellite positioning systems, such as the United States NAVSTAR Global Positioning System (GPS), the Russian Globalnaya navigatsionnaya sputnikovaya sistema (GLONASS), the Galileo global navigation system, the BeiDou Navigation Satellite System (BDS), the Compass global navigation system, the Indian Regional Navigation Satellite System (IRNSS), the Quasi-Zenith Satellite System (QZSS), etc.). For ease of illustration, embodiments will be described herein with specific reference to the GPS.

In the above or other embodiments, mobile computing device 103 makes use of a wireless triangulation/positioning system to determine the current location of the computing device and thus the current location of the cochlear implant 102. Wireless triangulation systems, sometimes referred to as Wi-Fi® positioning systems or indoor positioning systems, operate by measuring the intensity/strength of signals received from wireless access points. Wi-Fi® is a registered trademark of the Wi-Fi Alliance.

For example, due to the extensive use of wireless access points in urban areas, buildings, etc., mobile computing device 103 may, at any given time, receive signals from a plurality of access points. The mobile computing device 103 measures the strength of the signals received from the access points and generates, for example, received signal strength indicator (RSSI) values for each access point. Since the locations of the wireless access points are known and/or predetermined, the wireless triangulation system uses the measured strength of the signals received at the mobile computing device 103 to determine the location of the mobile computing device (i.e., triangulate the position of the mobile computing device relative to the position of the access points). Wireless triangulation systems are known in the art and are not described further herein.

In embodiments using satellite positioning systems, wireless triangulation systems, or other systems to determine a current location of the mobile computing device 103, it is assumed that the mobile computing device 103 is generally within the immediate proximity of the recipient such that the current location of the computing device 103 also represents the current location of the cochlear implant 102. It is to be appreciated that certain embodiments may use a device pairing mechanism to ensure that the techniques are implemented only when the mobile computing device 103 is in proximity to the cochlear implant 102 (e.g., Bluetooth® pairing). Bluetooth® is registered trademark of the Bluetooth® Special Interest Group (SIG).

Embodiments using a satellite positioning system, wireless triangulation system, etc. represent techniques that correlate the real-time (i.e., current) position of the cochlear implant system 100 with a selected map location. Other embodiments presented herein may use predetermined/pre-set (i.e., non-real time) selected map locations. More specifically, a user determines that the cochlear implant system 100 will be, or is likely to be, used in a particular venue (e.g., concert hall, school, restaurant, sports stadium, etc.). Prior to the cochlear implant system 100 entering the venue, the user may identify a point within the venue as the selected map location.

In one such embodiment, software at the mobile computing device 103 or an associated device (e.g., second computing device, fitting system, remote control, etc.) displays a digital map to the user. The user can use the displayed digital map and/or related functionality to pre-set one or more selected map locations. For example, a user may enter one or more inputs to select a point within a venue displayed as part of the digital map. In a further embodiment, software at the mobile computing device 103 or an associate device allows a user to select and/or input the name, GPS coordinates, or some other identifier for a venue. The software uses the entered venue identifier to determine that a selected map location is situated within the venue.

Returning to FIG. 4, after identification of a selected map location for the cochlear implant system 100, the method further includes, at 158, the analysis of digital map data to identify a bounded map area/region in which the selected map location is located/situated. That is, the digital map data for the selected map location is used to find edges in the digital map image which correspond to the edges of the venue (e.g., office building, school, sport stadium, golf course, restaurant, etc.) where the selected map location is situated. FIG. 5B is a schematic diagram illustrating a bounded map area 155 that corresponds to building 152 of FIG. 5A.

In certain embodiments, the bounded map area 155 is identified using one or more image processing techniques, such as edge detection. Edge detection refers to techniques that identify the boundaries of objects displayed as part of a digital image. Edge detection operates by detecting areas in which the image brightness changes sharply or, more formally, has discontinuities. The points at which the image brightness changes sharply are typically organized into a set of linear or curved segments termed “edges.” Edge detection techniques may include, for example, the computation of a Fourier transform of the map image and the performance of high pass filtering of the image, the use of a Gaussian smoothed step edge model, the use of Canny edge detection, the use of first and second order mathematical functions, the use of regularized cubic spline fitting, the use of color detection to determine roofs, the use of differential edge detection, the use of phase coherence and phase congruency, etc. Edge detection is known and further details thereof are not provided herein.

For example, applying edge detection to the example of FIG. 5A, the digital map 150 is analyzed to identify edges defining a bounded region around the selected map location 154. As shown in FIG. 5B, the detected edges correspond to the outline of displayed building 152. As such, the bounded map area 155 corresponds to the outer edges of building 152 displayed as part of digital map 150.

Edge detection represents an automated method that may be used alone or in combination with one or more other techniques to identify the bounded map area. In one illustrative embodiment, one or more user inputs (e.g., touch inputs, mouse/keyboard inputs, text, etc.) are entered to confirm, enlarge, reduce, etc., the size of a bounded map area determined using edge detection or another image processing technique. In alternative embodiments, only user inputs are used to identify the bounded map area. For example, user inputs may be entered to directly identify the corners or edges of the building 152 as the bounded map area.

In certain embodiments, limits may be placed on a possible maximum size for a bounded map area. Such limits may be useful, for example, if a user initiates the techniques in an overly large spatial region (e.g., an ocean) that would be too computationally expensive to define.

Returning to FIG. 4, after identification of the bounded map area 155 that encompasses the selected map location 154, the method further includes, at 160, generating/computing an aggregate mapped hearing area. As noted above, an aggregate mapped hearing area represents a spatial region in which the cochlear implant 102 is to activate and use one or more sound processing settings. The hearing area is referred to as being an “aggregate” area because it is formed by a collection/aggregation of a plurality of discrete sound setting sub-regions organized as geometric primitives overlaying and substantially covering the bounded map area 155.

Location-aware devices are able to determine when they are within a specified proximity to a specific position point (e.g., within a radius of position point). As such, each discrete sound setting sub-region (i.e., each geometric primitive) represents a boundary around a selected position point. As described further below, entry into, or exit from, the sound setting sub-regions is detectable by the location-aware mobile computing device 103. Therefore, the mobile computing device 103 can, depending on whether or not the device is within a sound setting sub-region, notify the cochlear implant 102 to use specific sound processing settings.

More specifically, an aggregate mapped hearing area is generated by calculating multiple position points (e.g., multiple GPS points) within the bounded map area. Each position point is associated with a different geometric primitive that represents a sound setting sub-region. A sufficient number of position points and associated geometric primitives are computed so as to substantially cover (overlay) the entire bounded map area.

For example, FIG. 5C is a schematic diagram illustrating a first geometric primitive 157(1) generated for bounded map area 155. As shown, the geometric primitive 157(1) of FIG. 5C is associated with a positioning point 161(1) that corresponds to the selected map location 154. The geometric primitive 157(1) extends to the edges of the bounded map area 155. The use of a first geometric primitive associated with the selected map location 154 is merely illustrative and other embodiments may begin with alternative position points in the bounded map area 155. Additionally, the size and shape of the geometric primitive 157(1) is illustrative.

As noted above, an aggregate mapped hearing area is generated by calculating multiple position points and associated geometric primitives. FIG. 5D illustrates a completed aggregate mapped hearing area 159 generated from the bounded map area 155. In the example of FIG. 5D, the aggregate mapped hearing area 159 includes nine (9) geometric primitives 157(1)-157(9) each associated with a different position point 161(1)-161(9), respectively. The geometric primitives 157(1)-157(9) substantially cover the underlying bounded map area 155.

Different methods may be implemented to generate the aggregate mapped hearing area 159 using geometric primitives. In one specific embodiment, one or more centrally located points are identified and/or distances between the edges of the bounded map area 155 are determined to centrally locate the first geometric primitive 157(1) (i.e., place the first positioning point 161(1) at a central area of the bounded map area 155). The radius of the geometric primitive 157(1) is then iteratively extended until the primitive reaches the outer edge of the bounded map area 155. The additional geometric primitives 157(2)-157(9) may be added in a similar manner by locating their respective positioning point 161(2)-161(9) at, for example, the central points of areas not yet covered by an earlier geometric primitive.

In certain examples, the overlap between two geometric primitives is maintained below approximately 50%. Additionally, although FIGS. 5C and 5D illustrate the geometric primitives 157(1)-157(9) generally terminating at the edges of the bounded map area 155, in alternative embodiments one or more of the geometric primitives 157(1)-157(9) may extend outside of the bounded map area 155.

FIGS. 5C and 5D illustrate the use of generally circular geometric primitives. Although illustrative, the use of circular geometric primitives may be beneficial by capitalizing on the ability of a device to determine when the device is within a defined radius of a position point. However, alternative embodiments may use other polygonal geometric primitives (e.g., triangles).

In one method, a number of geometrical primitives such as lines, curves, shapes and polygons may be fit within the bounded map area 155 using, for example, a least mean square (LMS) method. In other words, alternative embodiments may fit geometric primitives to the bounded map area 155 using one or more mathematical expressions. In certain embodiments, vector graphics can be used to create paths through control points or nodes to represent the bounded map area.

As noted, each of the geometric primitives 157(1)-157(9) represents a separate sound setting sub-region in which cochlear implant 102 is configured to activate and use one or more selected sound processing settings. Since all of the geometric primitives 157(1)-157(9) are associated with the same region (i.e., bounded map area 155) the geometric primitives 157(1)-157(9) all have the same associated sound processing settings and are linked together. In other words, the various sound setting sub-regions defined by the plurality of geometric primitives are “aggregated” or “collected” into a larger single defined region for use of the same sound processing settings therein. This enhances user experience since the recipient can seamlessly use the same desired settings through the entire bounded map area 155, rather than in only the various discrete sound setting sub-regions (i.e., there is no need to change settings when the recipient moves between the sound setting sub-regions).

The embodiments described above use a plurality of geometric primitives to create the aggregate mapped hearing area. However, it is to be appreciated that an aggregate mapped hearing may be defined through the use of other techniques. For example, the position coordinates of the corners, outer edges, etc. of a bounded map area may be entered and/or determined. In such examples, the position coordinates are connected together to define a bounded region that represents an aggregate mapped hearing area.

As noted above, after an aggregate mapped hearing area is defined, the techniques select one or more sound processing settings for use in the aggregate mapped hearing area (step 140 of FIG. 2). Also as noted above, the real-time position of the cochlear implant system 100 may be used to identify a selected map location (real-time selected map location). However, other embodiments presented herein use a pre-set selected map location to define an associated aggregate mapped hearing area. In each of these embodiments, a user may manually select one or more sound processing settings for use or the one or more settings may be selected by the cochlear implant system 100, or a related device, for use within the aggregate mapped hearing area.

In certain embodiments, the one or more sound processing are selected and/or changed through analysis of the sound environment corresponding to an aggregate mapped hearing area. For example, data characterizing the sound environment is recorded and then analyzed so as to optimize the settings of the cochlear implant 102 or to change features in the cochlear implant or the mobile computing device 103 to provide better sound quality. In further examples, a user can pay a monthly fee, initiate an in-app purchase, etc. to obtain a feature which can be useful for the detected and analyzed environment (e.g., purchase and activate a specific wind noise algorithm).

In one embodiment, the digital map data may be used to determine if the aggregate mapped hearing area is an indoor or outdoor environment. The cochlear implant system 100 is then set with sound processing settings that are more appropriate for indoor or outdoor use, respectively. In another embodiment, the digital map data may be analyzed to detect the type or kind of building/area associated with an aggregate mapped hearing area. For example, identifier data (i.e., map labels, building names, etc.), which is generally incorporated as part of the digital map data, is used determine if the aggregate mapped hearing area is a concert hall, sports stadium, library, etc. This additional data is then used to select the sound processing settings for use in that particular environment (e.g., select music settings or give a notification/suggestion to the user to change to music settings when in a concert hall).

In one specific embodiment, previous estimated or selected sound processing are compared to present environmental settings. The present environment settings may include the estimation of the number of persons present in a room. If there are multiple persons in the room speaking, the device can make changes to the settings to accommodate the specific environment. In another example, the recipient can be provided with one or more audible signals informing the recipient of the environment.

Various settings may be changed based on the analysis of the current environment. For example, operation of the cochlear implant may be adjusted for the altitude of the current location and/or to adjust output levels depending on surrounding air pressure, detected reverberation, wind or echo, etc. In another example, an accelerometer is used to estimate the gravitation force and this can be used to make sound processing setting adjustments. In another example, the location detection is combined with language detection. If, as an example, the cochlear implant system detects China as a location and the Mandarin language, the settings for the cochlear implant (for example for the compressor) are adjusted to perform better with tonal languages. Such information may sent to another entity (e.g., manufacturer, clinician, etc.) to correlate optimized device operation with the environment of primary use.

Also as noted above, after an aggregate mapped hearing area is defined and the one or more sound processing settings associated therewith, the techniques presented herein determine when the cochlear implant system 100 is first correlated with the aggregate mapped hearing area (step 142 of FIG. 2). Once it is determined that the cochlear implant system 100 is within the aggregate mapped hearing area, the one or more selected sound processing settings are activated (or selected in real-time and activated).

In certain embodiments, the one or more sound processing settings are activated upon entry into, or prior to entering a mapped area. That is, the one or more sound processing settings are activated when the cochlear implant system 100 is “first” or “initially” correlated with the aggregate mapped hearing area. As noted above, cochlear implant system 100 may be correlated with the aggregate mapped area when the cochlear implant system is at least one of positioned in, substantially positioned in, anticipated to be positioned in, or within a defined proximity of the aggregate mapped area. For example, the system may determine that the cochlear implant 100 is moving quickly and routinely towards a predetermined aggregate mapped hearing area. In such an example, the cochlear implant system 100 determines that it is likely that the cochlear implant 100 will soon enter the predetermined aggregate mapped hearing area (i.e., anticipates entry into the predetermined aggregated mapped hearing area) and, as a result, the system adjusts the settings prior to entry into the predetermined aggregate mapped hearing area.

In general, it may be advantageous to initiate the adjustment of settings/modes of operation before entering the aggregate mapped area so that the settings are fully implemented upon entry into the area. For example, when the cochlear implant system 100 is moving towards an aggregate mapped area, the current settings of the system are adapted/converged towards the settings selected for the aggregate mapped hearing area so that they are fully adjusted when entering the area, thereby creating a smoother listening experience instead of jumping between settings. This also means that the exact location is not as critical and, instead the current settings are a blend of previous settings and the settings defined for the specific area when the medical device gets closer to the aggregate mapped hearing area.

It can also be considered that if the current location is estimated to have an accuracy of +−10 meters such that transitions from previous setting and the new settings occur more quickly. In one specific example, a transition between settings may begin when the cochlear implant system 100 is approximately +−100 meters away from an expected location.

In one example, if the cochlear implant system 100 is moving a speed of X meters per second towards a mapped area, the system can set a timer to make a change of settings at the estimate time of arrival. In this way, the change is activated by a timer and not when entering the area (i.e., when the cochlear implant system 100 is “time” correlated with the aggregate mapped area).

In certain embodiments, one or more sound processing settings may be activated when the cochlear implant system 100 is “de-correlated” from am aggregate mapped area. The cochlear implant system 100 is de-correlated from an aggregate mapped hearing area when, for example, the medical device first exits the aggregate mapped hearing, exits a defined proximity of the aggregate mapped hearing area, or when the it is determined that the medical device is moving away from the mapped area. For example, it may be advantageous to activate/re-activate one or more processing settings when exiting a mapped area.

In one specific example, the cochlear implant system 100 or other medical device is correlated with an aggregate mapped area when the cochlear implant system or medical device is estimated to be positioned in a specified physical proximity of the aggregate mapped area. In certain embodiments, geo-fencing techniques are used determine when the cochlear implant system 100 has entered (or entered into proximity of) an aggregate mapped hearing area. A “geo-fence” is a boundary (e.g., a radius around a positioning point or another defined area). Geo-fencing techniques may make use of satellite positioning systems, wireless positioning systems, etc. However, the use of geo-fencing techniques that rely upon wireless positioning systems (including cellular base station information, RSSI calculations, etc.) may be operationally less expensive (e.g., use less power) than geo-fencing techniques that rely upon satellite positioning systems.

When the location-aware cochlear implant system 100 enters or exits a geo-fence, the system receives a generated notification/event. This notification may include, for example, information about the location of the device, the geo-fenced area, etc. In the embodiments presented herein, the geometric primitives that form an aggregate mapped hearing area each operate as a geo-fenced area (i.e., the edges of each geometric primitive is a geo-fence). As such, a notification is received when the cochlear implant system 100 crosses into a geometric primitive. When such a notification is received, the cochlear implant system 100 checks to see whether the newly entered geometric primitive is linked to a previous geometric primitive (if one exists). If a link is identified, then the cochlear implant system 100 determines that the newly entered geometric primitive forms part of the same aggregate mapped hearing area as the previous geometric primitive. However, if no link is identified, then the cochlear implant system 100 determines that the system has entered a new aggregate hearing area.

No notifications are provided to the recipient when transitions occur between two geometric primitives within the same aggregate hearing area. However, when a new aggregate hearing area is entered, the recipient is notified and/or one or more different sound processing settings are activated. The notifications to the recipient may take a number of different forms. In general, the cochlear implant system 100 suggests to the recipient to activate and use the one or more sound processing settings associated with an aggregate mapped hearing area and the recipient activates the settings within one or more inputs (e.g., voice input, touch input, etc.). The manner of notification to the recipient could be via a private beep mechanism (e.g., a sequence of beeps that are heard internally by the recipient when the cochlear implant system is trying to notify the recipient about a setting change). Another mechanism that can be used to inform the recipient of a sound processing setting change could be by playing a segment of speech to the recipient internally. These speech segments could be a phrase informing the recipient which recommended settings should be selected or a phrase requesting the recipient to accept suggested settings. In further examples, the mobile computing device 103 may provide a visual or audible notification. It can also be considered that a light change of sound processing settings is implemented without recipient, while the change to a more dedicated program requires acceptance by the recipient.

The boundary coordinates for an aggregate mapped hearing area are saved and stored by the mobile computing device 103. Therefore, whenever the recipient enters the aggregate mapped hearing area in the future, the mobile computing device 103 will still select the correct sound processing settings.

In certain embodiments, the techniques provide a smooth transition between different sets of sound processing settings based how close the recipient is to an aggregate mapped hearing area. For example, the cochlear implant system 100 may be configured to activate a first set of sound processing settings when in a particular aggregate mapped hearing area. The mobile computing device 103 is configured to determine when the cochlear implant system 100 is close to, but not yet within, the particular aggregate mapped hearing area (i.e., is within a region that is proximate to the aggregate mapped hearing area). When the cochlear implant system 100 enters into the region proximate to the particular aggregate mapped hearing area, the mobile computing device 103 causes the cochlear implant 102 to activate a second set of sound processing settings. The second set of sound processing settings are selected so as to make the expected transition to the first set of sound processing settings less abrupt (i.e., smooth transition).

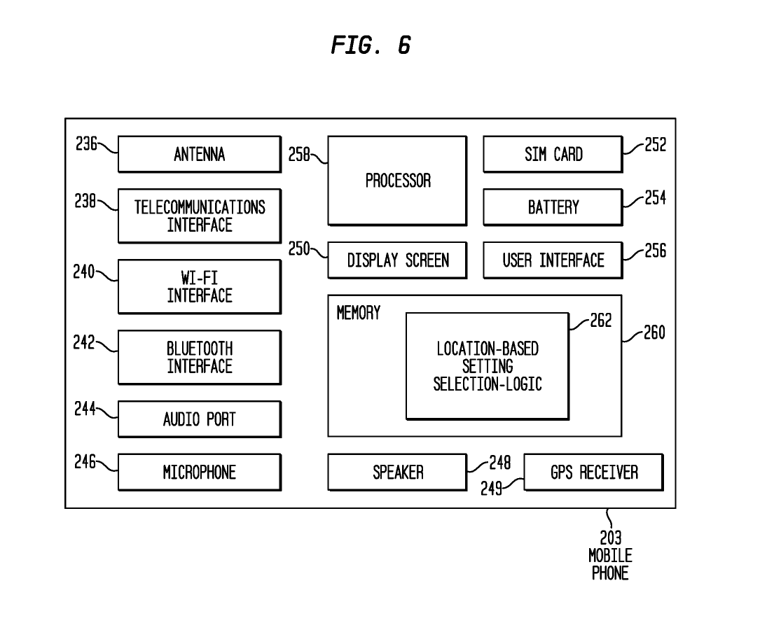

FIG. 6 is a block diagram of an illustrative arrangement for mobile computing device 103, referred to as a mobile phone 203. It is to be appreciated that FIG. 6 is merely illustrative of one arrangement for a mobile computing device configured to execute techniques for location-based selection of sound processing settings as described herein.

Mobile phone 203 comprises an antenna 236 and a telecommunications interface 238 that are configured for communication on a wireless communication network for telephony services. The wireless communication network over which the radio antenna 236 and the radio interface 238 communicate may be, for example, a Global System for Mobile Communications (GSM) network, code division multiple access (CDMA) network, time division multiple access (TDMA), or other kinds of networks.

As shown in FIG. 6, mobile phone 203 also includes wireless local area network interface 240 and an infrared (IR) or Bluetooth® interface 242. The wireless local area network interface 240 allows an electronic device to exchange data or connect to the Internet using, for example, 2.4 Gigahertz (GHz) Ultra high frequency (UHF) and/or 5 GHz Super high frequency (SHF) radio waves. The Bluetooth® interface 242 enables the mobile phone 203 to wirelessly communicate (i.e., directly receive and transmit data to/from another device via a wireless connection). In certain examples, the Bluetooth interface 242 may be used to wireless connect the mobile phone 203 to the cochlear implant 102 (FIG. 1). It is to be appreciated that the use of a wireless local area network interface in combination with a Bluetooth® interface is merely illustrative and that any other interfaces now known or later developed including, but not limited to, Institute of Electrical and Electronics Engineers (IEEE) 802.11, IEEE 802.16 (WiMAX), fixed line, Long Term Evolution (LTE), etc., may also or alternatively form part of the mobile phone 203.

Mobile phone 203 also comprises an audio port 244, one or more sound input elements, such as a microphone 246, a speaker 248, a display screen 250, a subscriber identity module or subscriber identification module (SIM) card 252, a battery 254, a user interface 256, a satellite positioning system receiver/chip 249 (e.g., GPS receiver), a processor 258, and a memory 260 that comprises location-based setting selection logic 262.

When conducting a voice call, speech signals received through antenna 236 and telecommunications interface 238 are analog to digital (A/D) converted by an A/D converter (not shown in FIG. 6), fed to audio part (also not shown in FIG. 2) that may be, for example, a codec configured to process signals under the control of processor 258. In certain embodiments, the signals are encoded so as to produce analog signals that are provided to speaker 248. The signals transmitted by speaker 248 may be detected by a microphone of the cochlear implant 102 for processing as described above for delivery of stimulation signals to the recipient (i.e., deliver stimulation signals representative of the speech signals received at the mobile phone 103). In alternative embodiments, the speech signals received by antenna 236 and telecommunications interface 238 are processed by mobile phone 203 and provided to the cochlear implant 102 through a wireless connection (e.g., via the Bluetooth® interface 242) or through a wired connection (e.g., via a connection between audio port 244 and a port of the cochlear implant 101). In still other embodiments, the mobile phone 203 is a telecoil-compatible phone so as to generate a magnetic signal representative of the speech signals which can be detected by a telecoil of the cochlear implant 101.

During a voice call, speech of the cochlear implant recipient may be detected at the microphone 246 of the mobile phone. After amplification and A/D conversion, the speech signals detected by the microphone 246 may be encoded and transmitted through telecommunications interface 238 and antenna 236.

The display screen 250 is an output device, such as a liquid crystal display (LCD), for presentation of visual information to the cochlear implant recipient. The user interface 256 may take many different forms and may include, for example, a keypad, keyboard, mouse, touchscreen, display screen, etc. In one specific example, the display screen 250 and user interface 256 are combined to form a touch screen. More specifically, touch sensors or touch panels have become a popular type of user interface and are used in many types of devices. Touch panels recognize a touch input of a user and obtain the location of the touch to effect a selected operation. A touch panel may be positioned in front of a display screen, or may be integrated with a display screen. Such configurations, allow the user to intuitively connect a pressure point of the touch panel with a corresponding point on the display screen, thereby creating an active connection with the screen. In certain embodiments, display screen 250 is used to provide a digital map for use during the location-based selection of sound processing settings described herein.

Memory 260 may comprise read only memory (ROM), random access memory (RAM), magnetic disk storage media devices, optical storage media devices, flash memory devices, electrical, optical, or other physical/tangible memory storage devices. The processor 258 is, for example, a microprocessor or microcontroller that executes instructions for the location-based setting selection logic 262. Thus, in general, the memory 260 may comprise one or more tangible (non-transitory) computer readable storage media (e.g., a memory device) encoded with software comprising computer executable instructions and when the software is executed (by the processor 258) it is operable to perform all or part of the presented techniques for location-based selection of sound processing settings in accordance with embodiments presented herein. That is, the location-based setting selection logic 262, when executed by processor 258 is a program/application configured to perform or enable the operations described herein at the mobile phone 203.

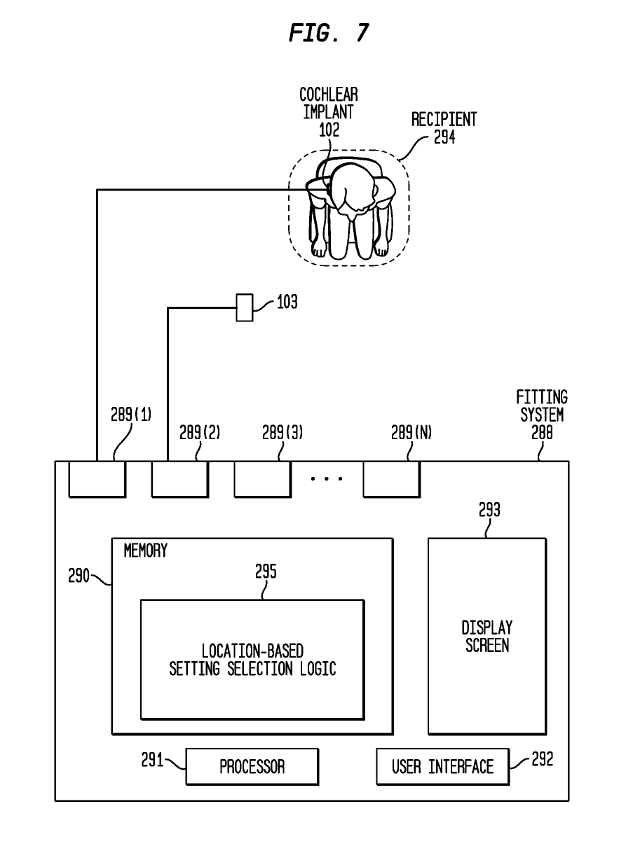

Embodiments have been primarily described herein with reference to a mobile computing device that operates perform all or part of the presented techniques for location-based selection of sound processing settings. However, it is to be appreciated that the techniques presented herein may be at least partially performed at another device that operates with a cochlear implant. For example, FIG. 7 is a block diagram of a fitting system 288 for use as part of the techniques for location-based selection of sound processing settings in accordance with embodiments presented herein. Fitting system 288 may be used, for example, to define an aggregate mapped hearing area and/or select sound processing settings for use in an aggregate mapped hearing area.

Fitting system 288 is, in general, a computing device that comprises a plurality of interfaces/ports 289(1)-289(N), a memory 290, a processor 291, a user interface 292, and a display screen 293. The interfaces 289(1)-289(N) may comprise, for example, any combination of network ports (e.g., Ethernet ports), wireless network interfaces, Universal Serial Bus (USB) ports, Institute of Electrical and Electronics Engineers (IEEE) 1394 interfaces, PS/2 ports, etc. In the example of FIG. 7, interface 289(1) is connected to cochlear implant 101 (FIG. 1) implanted in a recipient 294. Interface 289(1) may be directly connected to the cochlear implant 101 or connected to a device (e.g., remote control device, behind-the-ear processor, etc.) that is communication with the cochlear implant 101. In certain embodiments, interface 289(2) may be connected to an external device, such as mobile computing device 103, that forms part of cochlear implant system 100. Interfaces 289(1) and 289(2) may be configured to transmit/receive signals via a wired or wireless connection (e.g., telemetry, Bluetooth, etc.).

Memory 290 comprises location-based setting selection logic 295. Memory 290 may comprise any one or more of ROM, RAM, magnetic disk storage media devices, optical storage media devices, flash memory devices, electrical, optical, or other physical/tangible memory storage devices. The processor 291 is, for example, a microprocessor or microcontroller that executes instructions for the location-based setting selection logic. Thus, in general, the memory 290 may comprise one or more tangible (non-transitory) computer readable storage media (e.g., a memory device) encoded with software comprising computer executable instructions and when the software is executed (by the processor 291) it is operable to perform the fitting operations described herein in connection with the location-based setting selection logic 295.

The techniques presented herein have been primarily described and illustrated using two-dimensional (2-D) maps and 2-D polygonal geometric primitives (e.g., circles, ellipses, triangles, etc.). It is to be appreciated that the techniques presented herein may also be used with 3-D maps. In such embodiments, a bounded map area is defined in three dimensions (i.e., having a length, width, and a height). A 3-D aggregate mapped hearing area may be generated by filling the 3-D bounded map area with 3-D polygonal geometric primitives (e.g., cylinders, blocks, etc.) As such, reference to bounded map areas and aggregate mapped hearing areas refer to both 2-D and 3-D areas.

As noted, embodiments of the present invention have been primarily described herein with reference to a cochlear implant and, more particularly, to the location-based selection of sound processing settings for the cochlear implant. It is to be appreciated that the techniques presented herein are not limited to the adjustment of sound processing settings, but may also be used for the location-based selection of other settings of the cochlear implant. Additionally, it is to be appreciated that other medical devices may benefit from the use of different settings at different locations. As such, the techniques presented herein may be used for the location-based selection of various settings of other medical devices, such as other hearing prostheses (e.g., acoustic hearing aids, bone conduction devices, middle ear auditory prostheses, direct acoustic stimulators, electrically simulating auditory prostheses such as auditory brain stimulators and etc.), visual prostheses, sensors, pacemakers, drug delivery systems, defibrillators, functional electrical stimulation devices, catheters, air sensors or purifiers, hospital beds, patient identification (ID) badges or other hospital equipment, etc.

The invention described and claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed is:

1. A method, comprising:
defining an aggregate mapped area for a medical device, wherein the aggregate mapped area is a digital representation of a spatial region, and wherein the spatial region is defined through the analysis of digital map data and is associated with a selected location point;
selecting one or more processing settings for use by the medical device when situated in the aggregate mapped area;
determining that the medical device is at least one of positioned within, substantially positioned within, or is anticipated to be positioned within the aggregate mapped area; and
activating the one or more processing settings in response to the determining that the medical device is at least one of positioned within, substantially positioned within, or is anticipated to be positioned within the aggregate mapped area.

2. The method of claim 1, wherein defining the aggregate mapped area for the medical device comprises:
determining a bounded map area for the selected location point; and
computing a plurality of geometric primitives so as to substantially cover the bounded map area, wherein each of the geometric primitives represents a sub-region for use of the one or more processing settings by the medical device.

3. The method of claim 2, wherein computing the plurality of geometric primitives comprises:
computing a plurality of geometric circles each representing a radius around a corresponding position point for use of the one or more processing settings by the medical device.

4. The method of claim 1, further comprising:
identifying a current location of the medical device as the selected location point.

5. The method of claim 4, further comprising:
determining the current location of the medical device with a satellite location system.

6. The method of claim 4, wherein determining the current location of the medical device comprises:
determining the current location of the medical device with a wireless triangulation system.

7. The method of claim 1, further comprising:
identifying a location on a digital map as the selected location point.

8. The method of claim 1, wherein activating the one or more processing settings for the medical device comprises:
presenting a notification to a recipient of the medical device to activate the one or more processing settings.

9. The method of claim 1, wherein the medical device comprises a hearing prosthesis, the aggregate mapped area comprises an aggregate mapped hearing area, and the one or more processing settings comprise one or more sound processing settings.

10. A medical system, comprising:
a medical device; and
a computing device comprising a memory and one or more processors configured to:
determine a location for the medical system;
determine, based on the location of the medical system and map data, that the medical system is at correlated with an aggregate mapped area; and
activate one or more settings for the medical system in response to determining that the medical system is correlated with the aggregate mapped area.

11. The hearing system of claim 10, wherein the one or more processors are configured to:
define, through the analysis of digital map data and the current location, the aggregate mapped area; and
select the one or more processing settings for use by the medical device when situated in the aggregate mapped area.

12. The medical device of claim 11, wherein to define the aggregate mapped area, the one or more processors are configured to:
determine a bounded map area for the current location; and
computing a plurality of geometric primitives so as to substantially cover the bounded map area, wherein each of the geometric primitives represents a sub-region for use of the one or more processing settings by the medical device.

13. The medical device of claim 12, wherein computing the plurality of geometric primitives comprises:
computing a plurality of geometric circles each representing a radius around a corresponding position point for use of the one or more processing settings by the medical device.

14. The medical device of claim 12, wherein the one or more processors are configured to utilize a satellite positioning system to determine the current location of the medical device.

15. The medical device of claim 12, wherein the one or more processors are configured to utilize a wireless triangulation system to determine the current location of the medical device.

16. A system, comprising:
a memory; and
one or more processors configured to:
determine a selected map location for a medical device,
define an aggregate mapped area for the medical device, wherein the aggregate mapped area is a digital representation of a spatial region, and wherein the spatial region is defined through the analysis of digital map data and is associated with the selected map location, and
select one or more processing settings for use by the medical device when situated in the aggregate mapped area.

17. The system of claim 16, wherein the one or more processors are part of a medical system, and are further configured to:
determine when the medical device is correlated with the aggregate mapped area; and
activate the one or more processing settings when the medical device is correlated with the aggregate mapped area.

18. The system of claim 16, wherein to define the aggregate mapped area for the medical device, the one or more processors are configured to:
determine a bounded map area for the selected map location; and
compute plurality of geometric primitives so as to substantially cover the bounded map area, wherein each of the geometric primitives represents a sub-region for use of the one or more processing settings by the medical device.

19. The system of claim 18, wherein to compute the plurality of geometric primitives the one or more processors are configured to:
compute a plurality of geometric circles each representing a radius around a corresponding position point for use of the one or more processing settings by the medical device.

20. The system of claim 16, wherein the one or more processors are configured to identify a current location of the medical device as the selected map location.
